(19) United States
(12) Patent Application Publication
(10) Pub. No.: US 2002/0192738 A1
(43) Pub. Date: Dec. 19, 2002

(54) Tyrosinase assay

(76) Inventors: Janice Brissette, Charlestown, MA (US); Rong Han, Boston, MA (US); Lorin Weiner, Charlestown, MA (US)

Correspondence Address: LOUIS MYERS, Fish & Richardson P.C., 225 Franklin Street, Boston, MA, 02110-2804, US

(21) Appl. No.: 10/134,919

(22) Filed: Apr. 29, 2002

Related U.S. Application Data
(60) Provisional application No. 60/286,950, filed on Apr. 27, 2001.

(51) Int. Cl. G01N 33/53; C12Q 1/26

(52) U.S. Cl. 435/25; 435/7.5

(57) ABSTRACT

A novel assay for tyrosinase.

### RELATED APPLICATIONS

This application claims priority from U.S. Provisional Application Serial No. 60/286,950, filed Apr. 27, 2001, the contents of which are incorporated herein by reference in their entirety.

### BACKGROUND OF THE INVENTION

Tyrosinase (monophenol, 3, 4-dihydroxyphenylalanine: oxygen oxidoreductase, EC 1.14.1 8.1) is a copper-based oxidoreductase that catalyzes the rate limiting step in melanin synthesis, the hydroxylation of L-tyrosine to L-DOPA. In addition, tyrosinase catalyses the oxidation of L-DOPA to L-dopaquinone. Tyrosinase is generally exclusive to pigment producing cells (melanocytes) and is frequently unregulated in melanoma. The reaction products of the melanin biopathway include pigments such as dopachrome (red), indole 5, 6 quinone (purple or yellow), and melanin (brown). These pigments serve as the basis for fluorometric and calorimetric assays that detect tyrosinase activity (Hoal et al. (1982) Cancer Res 42:5191-5195; Buffey et al. (1994) Brit J Dermatology 131:836-842; Moore et al. (1989) a Histochemistry 90:379-381; PCT International Application No. WO 01/01131). Other tyrosinase detection techniques include radiometric assays for tyrosinase activity (Pomerantz (1964) Biochem Biophys Res Commun 16:188-192; Ramirez-Bosca (1992) Arch Dermatol Res 284:358-362) and antibody based detection techniques (Orchard (2000) Histochem J 32:475-481; Fetsch et al. (2000) Cancer Cytopathology 90:252-257; Wakisaka et al. (2000) Life Sciences 66:1-6). The dopa reaction has been the standard assay for tyrosinase activity in situ. In this procedure, a tissue sample, often an epidermal sheet, is incubated in dopa or tyrosine, producing a black pigment (dopa-melanin [3]) in tyrosinase-positive cells (Lerner & Hendee (1973) J Invest Dermatol 60, 16-19).

### SUMMARY OF THE INVENTION

The inventors have developed a highly sensitive and specific assay for tyrosinase, an enzyme essential for the production of melanin. The assay, described herein, includes detecting the presence or absence of a labeled reacted tyrosinase substrate. The assay can, e.g., show the in situ location of tyrosinase, e.g., within a cell or tissue; identify the presence of a tyrosinase containing cell, e.g., a pigment cell or melanoma cell, within a tissue, e.g., skin tissue, eye tissue, blood, serum, plasma, lymph; identify pigment cell distribution or pigment cell status within a tissue, e.g., skin or eye tissue. The assay works on frozen tissue sections, unfixed tissue, and works in conjunction with several types of fixatives. The assay can also work on paraffin sections, e.g., renatured paraffin sections.

Accordingly, the invention features a method of detecting tyrosinase in a sample, e.g., a tissue sample, e.g., in a skin, eye, or blood sample. The method includes: (a) contacting the sample with a tyrosinase substrate, e.g., tyrosine, tyramide, or DOPA, which substrate is coupled, directly or indirectly (e.g., through biotin-streptavidin) to a label, e.g., a calorimetrically detectable label (e.g., horseradish peroxidase or alkaline phosphatase) or a fluorescent label (e.g., fluorescein, Texas Red, or CY-3); (b) preferably allowing the tyrosinase to act on the substrate, e.g., to bind and/or to oxidize the substrate; and (c) detecting the presence or absence of the reacted labeled tyrosinase substrate. The signal can be detected through conventional techniques, e.g., fluorescence imaging, e.g., with a microscopic imager. While not wishing to be bound by theory, tyrosinase in the sample is thought to bind and/or oxidize the tyrosinase substrate, creating an unstable, reactive intermediate. The intermediate is thought to bond to nearby molecules and deposit onto the sample in close proximity to the tyrosinase, where it can be detected, or to incorporate into melanin.

In a preferred embodiment, the sample is a skin tissue sample.

In a preferred embodiment, the sample is an eye tissue sample.

In a preferred embodiment, the sample is a blood sample, e.g., blood, plasma or serum, or a lymph sample, e.g., a sample of lymphatic fluid.

In a preferred embodiment, the sample is a cultured cell or tissue sample.

In a preferred embodiment, the tyrosinase substrate is a phenolic compound.

In a preferred embodiment, the tyrosinase substrate is tyrosine or a tyrosine analog. In another preferred embodiment, the tyrosinase substrate is tyramide or DOPA.

In a preferred embodiment, the tyrosinase substrate, e.g., tyramide, DOPA, or a tyrosine analog, is coupled to biotin and the label is coupled to streptavidin.

In a preferred embodiment, the label is a fluorescent label, e.g., fluorescein, Texas Red, rhodamine, or CY-3.

In a preferred embodiment, the sample is a frozen section.

In a preferred embodiment, the sample is fixed in one or more of: methanol, acetone, or formaldehyde.

In a preferred embodiment, the sample is washed at least once, e.g., with PBS, e.g., to remove unreacted substrate or unbound label.

In a preferred embodiment, the reacted labeled substrate is bound to tyrosinase or to another molecule of the sample.

In a preferred embodiment, the method detects tyrosinase in situ in a tissue sample.

In another aspect, the invention features a method of detecting tyrosinase in a sample, e.g., a skin or eye tissue sample. The method includes: (a) contacting the sample with a tyrosinase substrate coupled to a first member of a specific binding pair, e.g., a biotinylated tyrosinase substrate, e.g., biotinylated tyramide or DOPA or a biotinylated tyrosine analog; (b) preferably allowing the tyrosinase to act on the substrate, e.g., to bind and/or oxidize the substrate; (c) contacting the sample with a second member of a specific binding pair, e.g., streptavidin, conjugated to a label, e.g., a colorimetrically detectable label (e.g., horseradish peroxidase or alkaline phosphatase) or a fluorescent label, e.g., fluorescein-streptavidin; and (d) detecting the presence of the label. In a preferred embodiment, the reacted labeled substrate is bound to tyrosinase or to another molecule of the sample. The presence of the label can be detected using conventional techniques, e.g., fluorescence imaging.

In a preferred embodiment, the sample is a skin tissue sample, e.g., a skin explant.

In a preferred embodiment, the sample is an eye tissue sample, e.g., an eye explant.

In a preferred embodiment, the sample is a cultured cell or tissue sample.

In a preferred embodiment, the sample is a blood sample, e.g., blood, plasma or serum, or a lymph sample, e.g., a sample of lymphatic fluid.

In a preferred embodiment, the tyrosinase substrate is a phenolic compound.

In a preferred embodiment, the tyrosinase substrate is tyrosine or a tyrosine analog. In another preferred embodiment, the tyrosinase substrate is tyramide or DOPA.

In a preferred embodiment tyramide is coupled to biotin and fluorescein is coupled to streptavidin.

In a preferred embodiment, the label is a fluorescein, Texas Red, rhodamine, or CY-3.

In a preferred embodiment, the sample is a frozen section.

In a preferred embodiment, the sample is fixed in one or more of: methanol, acetone, or formaldehyde.

In a preferred embodiment, the sample is washed at least once, e.g., with PBS, e.g., to remove unreacted substrate or unbound label.

In another aspect, the invention features a method of identifying a compound, e.g., a cosmetic, which modulates or affects pigmentation in the skin or hair, e.g., a compound that modulates melanogenesis in a melanogenic cell, e.g., a compound useful as a skin bleaching or skin darkening agent, or a sunscreen. The method includes: (a) contacting a cell or tissue with a test compound; (b) contacting the cell or tissue with a tyrosinase substrate, e.g., tyrosine, tyramide, or DOPA, which substrate is coupled, directly or indirectly (e.g., through members of a specific binding pair, e.g., biotin-streptavidin) to a label, e.g., a calorimetrically detectable label (e.g., horseradish peroxidase or alkaline phosphatase) or a fluorescent label, e.g., fluorescein, Texas Red, or CY-3; (c) preferably allowing the tyrosinase to act on the substrate, e.g., to bind and/or oxidize the substrate; and (d) detecting the presence or absence of the reacted labeled tyrosinase substrate. A compound that causes a change in the amount, localization, or distribution of tyrosinase in the cell or tissue can be identified as a compound that modulates melanogenesis. Preferably, the method detects tyrosinase in situ in a tissue sample. The signal from the label can be detected through conventional techniques, e.g., fluorescence imaging, e.g., with a microscopic imager. In some embodiment, the cell or tissue can be contacted with UV radiation, e.g., UVB radiation, in addition to, or instead of, a test compound.

In a preferred embodiment, the cell is an in vitro cultured cell, e.g., a melanocyte.

In a preferred embodiment, the tissue is a tissue explant, e.g., a skin explant.

In a preferred embodiment, the cell or tissue is a skin cell or tissue.

In a preferred embodiment, the cell or tissue is an eye cell or tissue.

In a preferred embodiment, the test compound is a small molecule, e.g., a small peptide or a small non-oligomeric molecule.

In a preferred embodiment, the test compound is an antibody or an antigen binding fragment thereof, such as Fab, Fab′₂, and Fv fragments.

In a preferred embodiment, the test compound is a plant extract or a plant derived compound.

In a preferred embodiment, the test compound is an organic compound.

In a preferred embodiment, the test compound is from a library of small molecules, e.g., small peptide or non-peptide molecules.

In a preferred embodiment, the test compound is from a library of antibodies.

In a preferred embodiment, the tyrosinase substrate is a phenolic compound.

In a preferred embodiment, the tyrosinase substrate is tyrosine or a tyrosine analog. In another preferred embodiment, the tyrosinase substrate is tyramide or DOPA.

In a preferred embodiment, the tyrosinase substrate, e.g., tyramide, DOPA, or a tyrosine analog, is coupled to biotin and the label is coupled to streptavidin.

In a preferred embodiment, the label is a fluorescent label, e.g., fluorescein, Texas Red, rhodamine, or CY-3.

In a preferred embodiment, the sample is a frozen section.

In a preferred embodiment, the sample is fixed in one or more of: methanol, acetone, or formaldehyde.

In a preferred embodiment, the compound is further tested in vivo on a human or non-human animal. For example, the compound is administered, e.g., topically, to the animal and the effect of the compound on the animal is evaluated.

In a preferred embodiment, the sample is washed at least once, e.g., with PBS, e.g., to remove unreacted substrate or unbound label.

In another aspect, the invention features a method of identifying a compound, e.g., a cosmetic, which modulates or affects pigmentation in the skin or hair, e.g., a compound that modulates melanogenesis in a melanogenic cell, e.g., a compound useful as any of: a therapeutic for a hyper- or hypo-pigmentary condition, a skin bleaching or skin darkening agent, or a sunscreen. The method includes: (a) contacting each of a plurality of cultured cells with a test compound, wherein each of the plurality is contacted with a different test compound; (b) contacting the plurality of cultured cells with a tyrosinase substrate, e.g., tyrosine, tyramide, or DOPA, which substrate is coupled, directly or indirectly (e.g., through members of a specific binding pair, e.g., biotin-streptavidin) to a label, e.g., a colorimetrically detectable label (e.g., horseradish peroxidase or alkaline phosphatase) or a fluorescent label, e.g., fluorescein, Texas Red, or CY-3; and (c) detecting the presence or absence of the labeled tyrosinase substrate associated with each of the plurality of cells. A compound that causes a change in the amount, localization, or distribution of tyrosinase in one or more of the plurality of cultured cells can be identified as a compound that modulates melanogenesis. The signal from the label can be detected through conventional techniques, e.g., fluorescence imaging, e.g., with a microscopic imager. In some embodiment, the cells can be contacted with a test treatment, e.g., UV radiation, e.g., UVB radiation, in addition to, or instead of, a test compound.

In a preferred embodiment, the cultured cells are melanocytes.

In a preferred embodiment, the test compound is a small molecule.

In a preferred embodiment, the test compound is a plant extract or a plant derived compound.

In a preferred embodiment, the test compound is an organic compound.

In a preferred embodiment, the test compound is from a library of test compounds, e.g., a library of small molecules, e.g., small peptide or non-peptide molecules; an antibody library; a library of organic compounds.

In a preferred embodiment, the tyrosinase substrate is a phenolic compound.

In a preferred embodiment, the tyrosinase substrate is tyrosine or a tyrosine analog. In another preferred embodiment, the tyrosinase substrate is tyramide or DOPA.

In a preferred embodiment, the tyrosinase substrate, e.g., tyramide, DOPA, or a tyrosine analog, is coupled to biotin and the label is coupled to streptavidin.

In a preferred embodiment, the label is a fluorescent label, e.g., fluorescein, Texas Red, rhodamine, or CY-3.

In a preferred embodiment, the cultured cells are fixed in one or more of: methanol, acetone, or formaldehyde.

In a preferred embodiment, the cultured cells are unfixed.

In a preferred embodiment, the compound is further tested in vivo on a human or non-human animal. For example, the compound is administered, e.g., topically, to the animal and the effect of the compound on the animal is evaluated.

In a preferred embodiment, the sample is washed at least once, e.g., with PBS, e.g., to remove unreacted substrate or unbound label.

In another aspect, the invention features a method of evaluating pigment cell status within a tissue, e.g., skin tissue, eye tissue, blood, plasma or lymph (e.g., lymphatic fluid) e.g., for the diagnosis or prognosis of a pigment cell disease, e.g., albinism, vitiligo, or a proliferative condition that involves pigment cells, e.g., melanoma. The method includes: contacting a tissue with a tyrosinase substrate, e.g., tyrosine, tyramide, or DOPA, which substrate is coupled, directly or indirectly (e.g., through biotin-streptavidin) to a label, e.g., a colorimetrically detectable label (e.g., horseradish peroxidase or alkaline phosphatase) or a fluorescent label, e.g., fluorescein, Texas Red, rhodamine, or CY-3; preferably allowing the tyrosinase to act on the substrate, e.g., to oxidize the substrate; and detecting the presence or absence of the reacted labeled tyrosinase substrate. In a preferred embodiment, the reacted labeled substrate is bound to tyrosinase or to another molecule of the sample. The presence or absence of tyrosinase correlates with pigment cell status in the tissue. Preferably, the method detects tyrosinase in situ in the tissue sample. The signal from the label can be detected through conventional techniques, e.g., fluorescence imaging, e.g., with a microscopic imager, e.g., by FACS or with a fluorescence microscope.

In a preferred embodiment, the tissue is a skin tissue.

In a preferred embodiment, the tissue is an eye tissue.

In a preferred embodiment, the tissue is a blood tissue, e.g., whole blood, plasma or serum, or a lymph tissue, e.g., a sample of lymphatic fluid or a lymph node, e.g., a lymph node biopsy.

In a preferred embodiment, the tyrosinase substrate is a phenolic compound.

In a preferred embodiment, the tyrosinase substrate is tyrosine or a tyrosine analog. In another preferred embodiment, the tyrosinase substrate is tyramide or DOPA.

In a preferred embodiment, the tyrosinase substrate, e.g., tyramide, DOPA, or a tyrosine analog, is coupled to biotin and the label is coupled to streptavidin.

In a preferred embodiment, the label is a fluorescent label, e.g., fluorescein, Texas Red, rhodamine, or CY-3.

In a preferred embodiment, the sample is washed at least once, e.g., with PBS, e.g., to remove unreacted substrate or unbound label.

In another aspect, the invention features a method of targeting a therapeutic agent to a pigment-positive cell, e.g., a melanoma cell. The method includes: administering to a cell, tissue or a subject in need thereof a tyrosinase substrate, e.g., tyrosine, tyramide, or DOPA, which substrate is coupled, directly or indirectly (e.g., through biotin-streptavidin) to a cytotoxic agent, e.g., ricin; saponin; pseudomonas exotoxin; pokeweed antiviral protein; diphtheria toxin; vinblastine; 4-desacetylvinblastine; vincristine; leurosidine; vindesine; an anti-metabolite such as cytosine arabinoside, fluorouracil, methotrexate or aminopterin; anthracyclines, mitomycin C; a vinca alkaloid; demecolcine; etoposide; mithramycin; an anti-tumor alkylating agent such as chlorambucil or melphalan; a DNA synthesis inhibitor such as daunorubicin, doxorubicin, adriamycin and the like.

In a preferred embodiment, the tyrosinase substrate is a phenolic compound.

In a preferred embodiment, the cell or tissue is a pigment cell cancer cell or tissue, e.g., a melanoma tissue.

In a preferred embodiment, the subject has or is at risk for a proliferative pigment cell disease, e.g., melanoma.

In another aspect, the invention features a method of targeting a therapeutic agent to a pigment-positive cell, e.g., a melanoma cell. The method includes: administering to a cell, tissue or a subject in need thereof, a tyrosinase substrate, e.g., tyrosine, tyramide, or DOPA, which substrate is coupled, directly or indirectly (e.g., through biotin-streptavidin) to a photosensitizer drug, e.g., a modified porphyrin, chlorin, bacteriochlorins phthalocyanine, naphthalocyanine, pheophorbide or purpurin; and exposing the cell, tissue or subject to light, e.g., a laser, whereby the photosensitizer drug becomes activated. Because the tyrosinase substrate targets the photosensitiser drug to a pigment positive cell (e.g., a melanoma cell present in the circulation), only pigment positive cells (e.g.,. melanoma cells in the circulation) are destroyed by the activated photosensitiser drug. Without being bound by theory, it is believed that the photosensitiser becomes activated by light, but it does not react directly with cells and tissues. Rather, it passes on its energy to molecular oxygen to form a particularly reactive toxic species called ‘singlet oxygen’.

In a preferred embodiment, the tyrosinase substrate is a phenolic compound.

In a preferred embodiment, the pigment positive cell is a melanoma cell.

In a preferred embodiment, the tissue is blood or lymph.

In a preferred embodiment, the subject has a pigment cell proliferative disease, e.g., melanoma.

As used herein, a “member of a specific binding pair” is each of two molecules that bind with specificity and high affinity to each other. Examples of a specific binding pair include biotin-streptavidin, or antigen-antibody.

As used herein, a “blood sample” or “blood tissue” refers to whole blood or to a sample or tissue derived from whole blood. For example, a blood sample or tissue includes plasma or serum. A “lymph tissue” refers to a tissue of the lymphatic system. For example, a lymph tissue includes a lymph node or a biopsy thereof, lymphatic fluid, or a lymphatic cell.

The details of one or more embodiments of the invention are set forth in the description below. Other features, objects, and advantages of the invention will be apparent from the description and drawings, and from the claims.

### BRIEF DESCRIPTION OF THE DRAWINGS

The patent or application file contains at least one drawing executed in color. Copies of the patent or patent application publication with color drawing(s) will be provided by the office upon request and payment of the necessary fee.

FIG. 1. Chemical structures of biotinyl tyramide and tyrosine The dashed line separates the tyramine and biotin groups of the substrate.

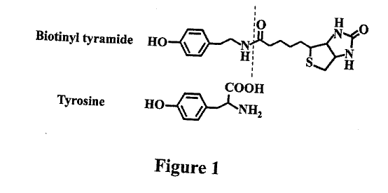

FIG. 2. Tyrosinase positive cells in human skin The tyrosinase assay (red) was performed on human skin fixed with formaldehyde (panels A, D, E, F) or methanol:acetone (panels B, C). DNA was counterstained with Hoechst dye 33258 (blue). Inset panels show melanin (black) stained by the Masson-Fontana technique. (Panels A, B, C, E) Normal skin. (Panel D) Skin with pigmented nevi. (Panel F) Skin with vitiligo. Arrowheads indicate the dermal/epidermal border. Scale bars for panels A, B, D-F, 50 μm; scale bar for panel C,25 μm.

FIG. 3. Tyrosinase assay and molecular markers of epidermal cells. Tyrosinase assay (red) and immunofluorescence (green) were performed on normal human skin fixed with formaldehyde. (Panels A-C) Comparison of tyrosinase-positive and c-Kit-positive cells; the three panels show the same field. (Panels D-F) Comparison of tyrosinase-and keratin-positive cells; the three panels show the same field. Arrowheads mark the dermal/epidermal junction. Scale bars, 50 μm.

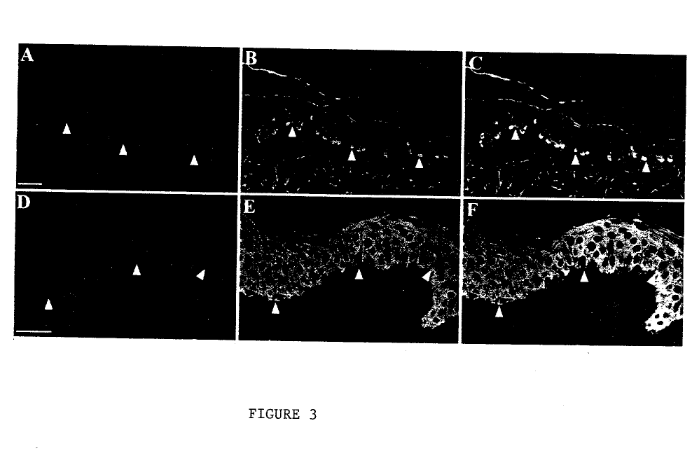

FIG. 4. Tyrosinase-positive cells in the adult eye. Formaldehyde-fixed eyes from six-month-old mice were stained using the tyrosinase assay (red) and Hoechst dye 33258 (blue) (Panels A, C) Black mice, strain C57BL/6. (Panels B, D) Albino mice, strain CD-1. I, iris; CH, choroid; RPE, retinal pigment epithelium Scale bar, 50 μm.

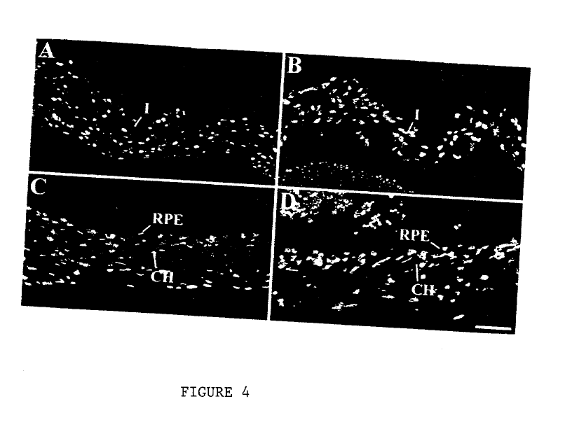

FIG. 5. Tyrosinase-positive cells in murine hair follicles Formaldehyde-fixed skin from seven-day-old mice was stained using tyrosinase assay (red) and Hoechst dye 33258 (blue) (Panels A, B) Black mouse, strain C57BL/6. (Panel C) Albino mouse, strain Swiss-Webster. In a panel B, tyrosinase assay was performed in the presence of kojic acid. MC, melanocyte cone; HS, hair shaft Scale bars, 50 μm.

FIG. 6. Tyrosinase activity in the hair shaft Murine skin was triple-stained using the assay described herein (red), antibodies to trichohyalin (green), and Hoechst dye 33258 (blue). The skin was derived from a seven-day-old mouse (strain C57BL/6) and fixed with formaldehyde. The three panels show the same field. (Panel A) tyrosinase and DNA. (Panel B) Trichohyalin and DNA. (Panel C) tyrosinase and trichohyalin. M, medulla; IRS, inner root sheath; MC, melanocyte cone. Scale bar, 25 μm.

FIG. 7. Epithelial tyrosinase activity. Formaldehyde-fixed skin from seven-day-old mice was stained for tyrosinase as described herein (Panel A; red) or with antibodies to S-100 (Panel B; red). DNA was counterstained with Hoechst dye 33258 (blue). In panel A, the arrow indicates the loss of tyrosinase and DNA staining. M, medulla; MC, melanocyte cone Scale bars, 50 μm.

### DETAILED DESCRIPTION

Human tyrosinase (monophenol, 3, 4-dihydroxyphenylalanine: oxygen oxidoreductase, EC 1.14.18.1) is an essential enzyme which regulates the production of melanin, a group of brown or black pigments present, e.g., in the skin and eyes of humans. More specifically, tyrosinase catalyzes the conversion of tyrosine to Dopa and of Dopa to dopaquinone. Tyrosinase is present in pigment producing cells (melanocytes) and is frequently unregulated in melanoma.

The invention features a specific, highly sensitive assay for tyrosinase activity in a cell or tissue. The method can detect tyrosinase e.g., in situ, e.g., in a skin or eye cell or tissue sample, or in vitro, e.g., in a cultured cell or tissue, e.g., a cultured melanocyte. The method includes: contacting the sample with a tyrosinase substrate, e.g., tyrosine, tyramide, or DOPA, which substrate is coupled, directly or indirectly (e.g., through biotin-streptavidin) to a label, e.g., a fluorescent label, e.g., fluorescein, Texas Red, or CY-3; and detecting the presence or absence of the reacted labeled tyrosinase substrate. Preferably, the tyrosinase substrate, e.g., tyramide, DOPA, or a tyrosine analog, is coupled to biotin and the label is coupled to streptavidin. The label can be a fluorescent label, e.g., fluorescein, Texas Red, rhodamine, or CY-3. The label can thus be detected through conventional techniques, e.g., fluorescence imaging.

The tyrosinase assay methods described herein are highly specific and sensitive. The methods do not require detecting colored pigments through bright field microscopy and do not generate background staining due to autooxidation of enzymatic reaction products. Instead, the use of a fluorescence-based visualization step produces a highly sensitive assay. For example, the methods described herein can detect active tyrosinase in, e.g., the medulla cells of the hair shaft.

By probing skin with the assays described herein, the inventors have identified a new site of tyrosinase activity—the epithelial cells of the hair medulla—which are known to receive numerous melanosomes from melanocytes. Given the key role of tyrosinase in melanin production, these results suggest that melanogenesis continues following melanosome transfer, conferring a pigmentary finction on cells that, by their nature, are not pigment forming.

Methodology

The tyrosinase assay described herein possesses similarities with catalyzed reporter deposition (CARD [35]), a signal amplification technique applied to immunostaining and other immunoassays In the CARD procedure, horseradish peroxidase is attached to a solid phase (e.g., a tissue section), often through linkage of the enzyme to an antibody or streptavidin The peroxidase subsequently reacts with a biotinylated phenolic compound (eg biotinyl tyramide), causing the deposition of this compound near the enzyme. Presumably, the peroxidase converts the phenol group into a free radical, which bonds to electron-rich molecules on the solid phase surface The solid phase is then probed with streptavidin conjugated to either a dye or an enzyme These streptavidin conjugates bind to the biotin deposits, thereby amplifying the immunoassay signal.

The tyrosinase assay described herein takes advantage of the structural resemblance between the natural substrates of tyrosinase and other phenolic compounds, e.g., biotinyl tyramide. In one embodiment of the assay, histological sections are incubated with biotinyl tyramide, typically for about 10 minutes. Analogous to CARD, the assay generates stable deposits of the biotinylated substrate, which are then detected with streptavidin-dye conjugates.

During staining, peroxidases are a potential source of background, since these enzymes may react with the biotinyl tyramide, causing CARD-like deposition. Thus, at the start of the assay procedure, can be exhausted or quenched by treatment with hydrogen peroxide.

Tyrosinase possesses greater resistance to peroxide treatment, maintaining its activity at peroxide concentrations that exhaust peroxidase. Thus, the assay procedure can utilize peroxide treatment to enhance assay specificity.

Tyrosinase Substrates

The methods described herein involve contacting the cell or tissue sample to be assayed (e.g., the cell or tissue explant or an in vitro cultured cell or tissue) for tyrosinase activity with a tyrosinase substrate. The tyrosinase substrate can be, e.g., tyrosine, tyramide, or DOPA (3 hydroxyltyrosine), or a tyrosine analog or derivative or other phenolic compound which is capable of reacting with tyrosinase. Preferably, the substrate is coupled to biotin.

Among the compounds which are substrates for tyrosinase which can be used are, e.g., dopamine, resorcinol, 4-hydroxyanisole, butylated hydroxyanisole, L-3,4-dihydrophenylalanine, tertbutylcatechol, hydroquinone, 6-hydroxydopa, N-acetyl-4-S-cysteaminylphenol (N-Ac-4-S-CAP) or methyl gallate.

Processing of Samples

The assays described herein are not restricted in their use to separated epidermis. The assays also work frozen tissue sections, e.g., intact skin and eye sections, and are therefore useful for histological analysis and clinical diagnosis. The methods do not require a particular method of tissue fixation, as the assay works with unfixed cells or tissue or with several kinds of fixatives, e.g., methanol/acetone fixation, or formaldehyde fixation. The assay can work with paraffin sections, e.g., renatured paraffin sections. Other useful tissue fixation methods are known to one of ordinary skill in the art.

Biotinylation of Proteins

Biotin is a small molecule which can have little effect on a molecule's function or even molecular weight. Biotin's small size makes it an excellent tag in the methods described herein. Biotin binds tightly to avidin or streptavidin. Avidin and streptavidin are available from commercial sources (e.g., Molecular Probes, Inc.) already conjugated to labels. Methods of making biotinylated compounds are described in Avidin-Biotin Chemistry: A Handbook, Pierce Chemical Company, 1992. The primary building blocks for preparing biotinylation reagents are biotin and biotin-XX, where “X” represents a seven-atom aminohexanoyl spacer between biotin and the reactive carboxylic acid. This spacer helps to separate the biotin moiety from its point of attachment, potentially reducing the interaction of biotin with the biomolecule to which it is conjugated. Avidin and streptavidin are generally interchangeable.

Preferably, the tyrosinase substrate, e.g., tyramide, DOPA, or a tyrosine analog, is coupled to biotin and the label is coupled to streptavidin. Biotinyl tyramide and streptavidin coupled to fluorescent labels, e.g.., streptavidin-fluorescein or streptavidin-Texas Red, are available commercially. Biotinyl tyramide can also be prepared as described herein (see Example 3).

Uses

The methods described herein will be useful in diagnostic, prognostic, and screening applications. The color of mammalian skin and hair is determined by a number of factors, including the degree of tyrosinase activity, which is the key and rate limiting enzyme for melanin production. Melanin is found in specialized pigment producing cells known as melanocytes. These cells originate in the neural crest and during embryogenesis are distributed throughout the body, including the skin, eyes, and CNS. Those that are present in the skin are normally present in the basal layer of the epidermis and the hair follicles. Thus, the presence or absence of tyrosinase in these tissues, assayed by the methods described herein, can be used, for example, in the diagnosis of pigment cell diseases, e.g., hyper- or hypo-pigmentary conditions or disorders, e.g., vitiligo or albinism, e.g., tyrosine-negative oculocutaneous albinism, or tyrosinase-positive oculocutaneous albinism.

Melanoma cells also express tyrosinase. Thus, the methods described herein can also aid in the diagnosis of melanoma. For instance, the assay can be used for the identification of circulating tyrosinase-expressing tumor cells in melanoma patients. Also, since tyrosinase is not normally found in lymph nodes or in the circulation, the presence of tyrosinase (and by extension, melanin containing cells) in lymph node sections or blood samples can be used as evidence that metastatic melanoma cells are present. In addition, melanomas expressing early markers but lacking intermediate or late markers have an epithelial morphology, lack pigmentation, and have low levels of tyrosinase. In contrast, melanomas expressing late markers have a spindle-shaped or polydenritic morphology, are pigmented, and have high levels of tyrosinase. Thus, the tyrosinase assays described herein can be useful to distinguish between these different stages.

The methods is particularly useful as a high-throughput assay for known or potential drugs or treatments (test compounds or treatments), e.g., cosmetics, that affect pigmentation, e.g., potential skin, eye, or hair pigmentation or de-pigmentation compounds. For example, in one embodiment, one can provide a plurality of cells, e.g., cultured cells, e.g., cultured melanocytes, and screen compounds (e.g., botanical compounds or plant extracts, or a library of test compounds, e.g., small molecules) for their ability to modulate tyrosinase activity in the cultured cells. The tyrosinase activity of the cells can be assayed (e.g., in the presence and absence of the test compounds) according to the methods described herein. Test compounds that affect (e.g., increase or decrease) the tyrosinase activity of the cultured cells can be identified as drugs or treatments for the treatment of hyper- or hypo-pigmentary conditions, e.g., conditions or disorders described herein, or as cosmetic treatments for pigmenting or de-pigmenting tissue, e.g., skin, hair, or eyes. In addition, since UV light stimulates tyrosinase activity, the assays described herein can test the effectiveness of sunscreens or other agents that protect against UV damage.

Pigmentation of Skin

Using the assay described herein, tyrosinase positive cells were detected at regular intervals in the basal layer of the epidermis. These cells appeared dendritic, possessing stained processes, or rows of stained granules, that extended between other epidermal cells. Thus in location and morphology, the tyrosinase-positive cells resembled melanocytes. It is thought that melanocytes vary in number according to body site, with the ratio of melanocytes to basal keratinocytes ranging from 1:4 to 1:10 [8]. The number of tyrosinase-positive cells fell within these melanocyte estimates, though staining generally matched the highest reported densities of melanocytes. Thus, the frequency of tyrosinase-positive cells was consistent with the accepted distribution of melanocytes.

As part of assay development, the assay was tested on tissue fixed with formaldehyde or methanol:acetone. The assay worked with both fixatives, as positive cells displayed intense signals and similar distributions in all samples. However, a lack of fixation did not impede staining, as positive cells exhibited similar frequencies in fixed and unfixed human skin. The assay stained unfixed skin even when the tissue was maintained in PBS for several hours at 4° C. prior to embedding. Thus, the assay detects a highly stable feature of a specific cell population and, as a result, does not require a particular method of tissue preparation or fixation.

To assess sensitivity, the assayed skin samples were stained for melanin using the Masson-Fontana technique. Melanin was abundant in one biopsy and barely detectable in the other, yet the two samples displayed equivalent tyrosinase staining patterns in the assay describe herein. Thus, the assay is not dependent on the level of melanin and produces a strong signal in skin with little pigmentation.

As a test of the assay's efficacy, the assay was performed on skin with pigmented nevi, the benign melanocyte tumors commonly known as “moles” [1]. Positive cells were found in clusters at the dermal-epidermal junction, and these clusters resembled melanocyte nests, structures characteristic of nevus histology. Thus, the assay is capable of identifying pigment cell pathologies, including melanocytic tumors.

To confirm the enzymatic basis of the assay, normal skin was assayed in the presence of kojic acid, an inhibitor of tyrosinase [37]. This inhibitor completely blocked the staining reaction, confirming that tyrosinase generates the signal. As a further test of assay specificity, the assay was performed on skin diagnosed with vitiligo, a disease resulting in the loss of cutaneous melanocytes [1]. No staining was observed in vitiligo-affected skin, showing that the assay identifies a trait strictly associated with pigment cells.

In one embodiment of the assay, biotinyl tyramide is applied to the sections in a buffer developed for CARD (amplification diluent; see Examples), which contains a low concentration of hydrogen peroxide [35]. To assess peroxide's role, the amplification diluent was replaced by 50 mM Tris (pH 8 0) with or without 0 01% hydrogen peroxide. In assays of normal human skin, positive staining was visible without peroxide, but peroxide greatly amplified the signal, increasing the sensitivity of the assay (data not shown). Thus, the assay can be performed with a common buffer, and hydrogen peroxide facilitates or stimulates the staining reaction.

It is thought that melanocytes are the sole producers of tyrosinase in the skin, but since the enzyme localizes to melanosomes, epithelial cells may acquire tyrosinase during pigment transfer. Thus, to identify all tyrosinase positive cell types, normal skin was double-stained using the assay described herein and antibodies to either melanocyte or keratinocyte markers. In the epidermis, tyrosinase staining correlated precisely with the distribution of c-Kit, a receptor tyrosine kinase present on melanocytes and several nonepidermal cell types (e.g., mast cells, germ cells, and hematopoietic stem cells [38]). In contrast, the tyrosinase staining pattern did not overlap with the distribution of keratin, the intermediate filament proteins characteristic of epithelial cells. Thus, in the epidermis, assay-positive cells exclusively possess markers associated with melanocytes. Thus, the assay described herein is a specific indicator of pigment cells in the skin, and tyrosinase is the likely catalyst of the staining reaction. The assay is highly sensitive, as it detects numerous melanocytes in skin with low melanin levels.

Pigmentation in the Eye

As a further test of its effectiveness, the assay was performed on eyes from black or albino mice. The albino animals carry a missense mutation in the tyr gene [29, 31], which inactivates tyrosinase and abolishes melanin synthesis. In adult black mice, strong staining was observed throughout the iris and choroid, precisely matching the distribution of melanin. In contrast, the retinal pigment epithelium exhibited weak staining, though this compartment also possessed significant melanin levels. Kojic acid inhibited all ocular staining, identical to its effect on human skin assays In albino animals, weak staining was observed in the retinal pigment epithelium, but no staining was detected in the iris and choroids.

Taken together, these results demonstrate that the tyrosinase assay is a highly specific indicator of tyrosinase activity. In the iris and choroid, the assay displays an absolute specificity for tyrosinase, as the albino mutation eliminates all staining In the retinal pigment epithelium, weak staining is generated through a tyrosinase-independent mechanism (perhaps the tyrosinase-related proteins [2, 19]), since this staining was not prevented by the albino mutation

Nonetheless, this weak signal differs dramatically with the intense staining specific to tyrosinase, and thus, tyrosinase is the only source of a strong signal.

It is known that ocular melanogenesis is greatest in early life, and that the adult eye exhibits a low rate of melanin turnover ([39] and references therein). Using 3-H-methimazole incorporation as a marker for melanin production, Lindquist et al. [39] probed mature murine eyes and detected melanogenesis in the iris and choroid; for adult as well as juvenile animals, no melanin synthesis was observed in the retinal pigment epithelium. Consistent with this study, the assay described herein detects significant tyrosinase activity in the iris and choroid, but little or no activity in the retina. Thus in adult mice, the retina normally lacks the ability to produce melanin, while the iris and choroid maintain their pigment-forming function.

Pigmentation of Hair

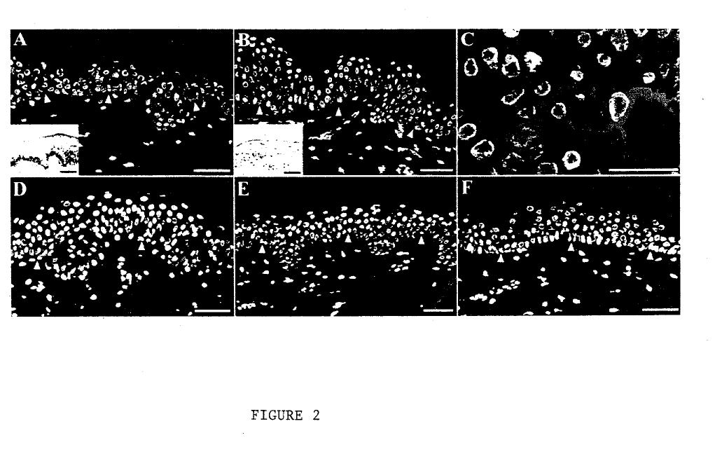

To examine melanogenesis in the hair follicle, the methods described herein were performed on skin from seven-day-old black mice. At this developmental stage, the skin contains a high density of hair follicles producing pigmented hair. Tyrosinase staining was most intense near the base of the differentiating hair shaft (FIG. 2), the site of most melanocytes in murine skin. These tyrosinase-positive cells formed a cone around the apex of the follicular papilla, which resembled the conical melanocyte cluster known to pigment the hair. Surprisingly, staining was not limited to the melanocyte cone, as tyrosinase-positive cells were observed within the differentiating hair itself. This hair-shaft staining proceeded from the cone towards the surface of the skin and exhibited a lower intensity than the conical signal. While most cutaneous staining was associated with the growing hair, positive cells were also detected occasionally in the epidermis, dermis, and outer root sheath, consistent with reports of melanocytes or their precursors in these compartments [6].

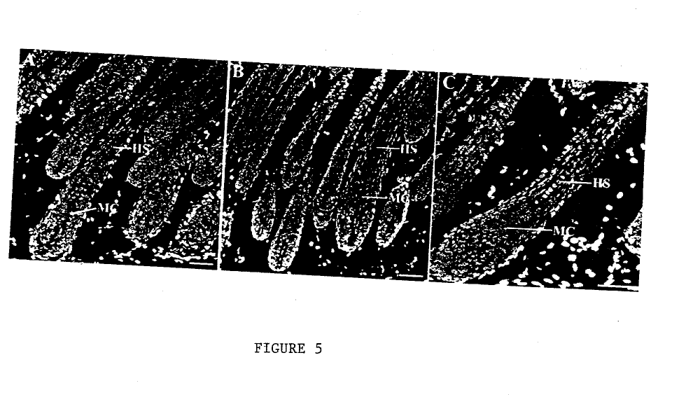

As in other pigmented tissues, kojic acid blocked tyrosinase staining in the skin of black mice (FIG. 5B). Moreover, albino animals exhibited no positive cells in the hair follicles (FIG. 5C), dermis, or epidermis (not shown). Thus, the assay is specific for tyrosinase activity in the skin and its appendages. Within the hair shaft, melanin normally accumulates in the cortex and (if present) medulla, which form concentric cylinders [10]. In mouse coat hair, the medulla typically exhibits greater pigmentation than the cortex, and within the medulla, the melanin becomes concentrated at regular intervals, producing a ladder of pigmented bands.

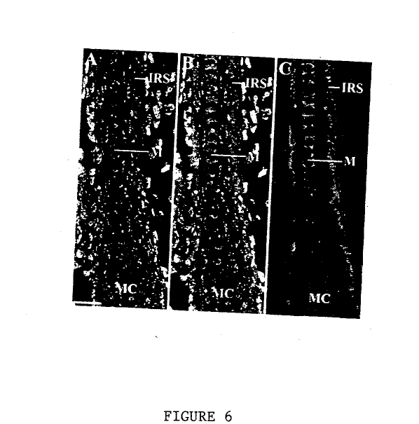

To assess the location of tyrosinase in the hair shaft, skin was triple-stained using the tyrosinase assay, Hoechst dye 33258, and antibodies to trichohyalin, a marker of the medulla and inner root sheath [40, 41]. As shown in FIG. 6, positive cells were found mainly in the medulla, but the tyrosinase staining did not overlap with the trichohyalin or DNA staining. Rather, the tyrosinase and trichohyalin/DNA signals generated a ladder of alternating bands, showing that tyrosinase, like melanin, is sequestered into a distinct compartment.

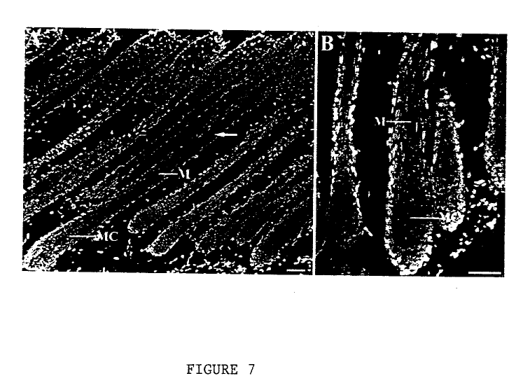

Based on electron microscopy and other studies, it is thought that melanocytes remain anchored in the hair bulb during the growth of a hair [6, 9, 10]. At the base of the hair shaft, melanocytes transfer melanosomes to the precursors of the cortex and medulla, which migrate upwards through the conical melanocyte cluster; for normal hair follicles, there is no evidence that melanocytes rise with the epithelial cells of the growing hair Consistent with this idea, staining proceeded from the hair bulb to the midpoint of the follicle, where its disappearance coincided with the loss of nuclei by medullary epithelial cells (FIG. 7A). It is known that the destruction of the nucleus is one of the final steps in the differentiation of the medulla and other cutaneous epithelia [9, 10]. Thus, tyrosinase activity is lost as the epithelial cells complete their differentiation and die, suggesting that these cells possessed the tyrosinase.

To examine dendritic cell distribution directly, skin was stained using antibodies against S-100, a protein present in melanocytes, Langerhans cells, neural cells, and other cell types [42]. In the internal portions of the hair follicle, S-100 was detected in a conical region around the follicular papilla (FIG. 7B), matching the accepted location of melanocytes. No S-100 staining was observed in the differentiating hair shaft, consistent with the absence of melanocytes from this structure. Thus, it can be concluded that the hair follicle contains active tyrosinase in two cell types—melanocytes and the differentiating epithelial cells of the medulla.

### EXAMPLES

### Example 1

### Tissue Processing

Prior to staining, tissue samples can be processed in different ways. The methods described herein work with frozen sections and several kinds of fixatives, e.g., methanol/acetone fixation, or formaldehyde fixation, with paraffin sections, e.g., renatured paraffin sections, and with unfixed tissue. The processing of tissue described herein is representative and is not meant to be limiting. Various other fixation and processing techniques are known to those of ordinary skill in the art. The method works without fixation as well.

For formaldehyde fixation, tissue samples were incubated overnight at 4° C. in phosphate-buffered saline (PBS) with 1% methanol-free formaldehyde (Polysciences, Inc) The samples were then transferred to 20% sucrose at 4° C. for 7-24 hours. To embed the tissue for sectioning, biopsies were blotted briefly on lens paper (to remove excess sucrose solution) and surrounded with OCT compound (Tissue-TekNVWR Scientific) in a peel-a-way tray (VWR Scientific) The tissue was then flash-frozen in an isopentane bath at −70° C. Sections were cut at a thickness of ˜6 μm, air-dried at room temperature, and stored at −70° C.

For methanol:acetone fixation, tissue samples were flash-frozen in OCT compound immediately after biopsy Sections were cut at a thickness of ˜6 μm and, after adherence to the slide, placed directly (while wet) in 1:1 methanol:acetone at ˜20° C. In general, samples were fixed for 5-15 minutes, but the length of fixation did not affect results Following fixation, sections were air-dried at room temperature and stored at −70° C.

To observe the distribution of melanin, Masson-Fontana staining [34] was performed.

### EXAMPLE 2

### Visualization of Tyrosinase

The following is one embodiment of the methods described herein.

All steps of the procedure were performed at room temperature in a humidified chamber. Formaldehyde-fixed sections were permeabilized with 0 1% NP-40 in PBS for 15 min; methanol:acetone-fixed sections were hydrated with PBS for 5 min and did not require permeabilization To quench peroxidase activity, all sections were treated with 3% H₂O₂ in PBS for 10 min; peroxide was then removed by one wash with PBS (5 min). To reduce background staining, the samples were blocked with 5% bovine serum albumin (BSA; fraction V; Boehringer Mannheim) in PBS (10-30 min); this incubation was followed by treatment with the avidin/biotin blocking kit of Vector Laboratories

The tyrosinase reaction utilized biotinyl tyramide and amplification diluent from the TSA Biotin System (NEN Life Science Products, Inc ), a kit optimized for the CARD (catalyzed reporter deposition) staining technique (35). (One can also use biotinyl tyramide prepared as described in Example 3). The biotinyl tyramide was reconstituted in dimethyl sulfoxide (DMSO) according to the manufacturer's instructions, diluted 1:50 in amplification diluent, and applied to the sections For murine samples, the tyrosinase reaction was incubated 5-10 min, while for human samples, the incubation time was 10-20 min. The sections were then washed three times with 0.1% NP-40/PBS (5 min each wash). Strepta-,idin-CY3 was diluted into 5% BSA/PBS (1:600) and incubated with the sections for 1 hour The samples were then washed once with 0 1% NP-40/PBS (5 min). Hoechst dye 33258 (10 mg/ml in PBS; Fluka Chemical Corp ), a DNA stain, was diluted 1:10000 into 0 1% NP-40/PBS and applied to the sections for 2 min The samples were washed once with 0 1% NP-40/PBS (5 min) and once briefly with distilled water. The sections were then air-dried and mounted with fluorescence mounting medium (Kirkegaard and Perry). Visualization of the samples can be performed through conventional fluorescence imaging techniques.

### EXAMPLE 3

### Preparation of Biotinylated Tyramide

Stock solution:

Sulpho-NHS-LC biotin (100 mg)

50 mm borate buffer pH8.0 (40 ml)

Tyramide hydrochloride (30 mg)

Stir gently at room temperature until solution completely dissolved. Filter through 0.45 μm syringe filter.

Working solution:

Stock solution (25 μl)

0.05M TBS pH 7.6 (1 ml)

Aliquot and store at −20° C.

### EXAMPLE 4

### Immunofluorescence

Immunofluorescent staining was performed as described [36], except that frozen sections were permeabilized with 0 1% NP-40/PBS (15 min) at the start of the procedure Rabbit polyclonal antibodies to S-100 (1:500) were from Neomarkers, Inc/Lab Vision Corp.

To double-stain sections using the methods described herein and immunofluorescence, the tyrosinase assay protocol was performed up to (but not including) the streptavidin-CY3 incubation Primary antibodies (rabbit polyclonals) were then diluted into 5% BSA/PBS and applied to the sections for 1 hour at room temperature. Antibodies to human c-Kit (1:100) were from MBL, antibodies to pan-cytokeratin (1:50) were from Zymed, and antibodies to trichohyalin were the gift of Dr. George E. Rogers (University of Adelaide). Following this incubation, the sections were washed three times with 0 1% NP-40/PBS (5 min). StreptavidinCY3 (1:600) and fluorescein-conjugated goat antibodies to rabbit IgG (1:50; Pierce) were diluted together into 5% BSA/PBS and applied to the sections for 1 hour at room temperature. The sections were then washed with 0 1% NP-40/PBS, stained with Hoechst dye 33258, washed again and mounted as described herein.

### REFERENCES

1. Bleehen (1998) Disorders of skin colour In “Rook/Wilkinson/Ebling Textbook of Dermatology” (Champion, Burton, Burns, and Breathnach, eds), pp 1753-1815, Blackwell Science Ltd, Oxford.

2. Hearing and Tsukamoto (1991) Enzymatic control of pigmentation in mammals Faseb J 5, 2902-2909

3. Prota (1988) Progress in the chemistry of melanins and related metabolites Med Res Rev 8,525-556

4. Masson, P (1948) Pigment cells in man In “The Biology of Melanomas” (R. W. Miner, and M Gordon, eds), Vol 4 pp 15-37, New York Academy of Sciences, New York

5. Rawles, M. E. (1948) Physiol Rev 28, 383-408.

6. Hirobe, T (1995) Histol Histopathol 10, 223-237

7. Scheie, H. G. , and Albert, D. M. (1977) “Textbook of Ophthalmology”, W. B. Saunders Co, Philadelphia

8. Ross, M. H., Romrell, L. J., and Kaye, G. I. (1995) “Histology: A Text and Atlas”, Williams & Wilkins, Baltimore

9. Zelickson, A. S. (1967) “Ultrastructure of Normal and Abnormal Skin”, Lea & Febiger, Philadelphia

10. Chase, H. B. (1954) Physiol Rev 34,113-126

11. Fitzpatrick, T. B. ,Miyamoto, M, and Ishikawa, K (1967) Arch Dermatol 96, 305-323

12. Seiji, M, Fitzpatrick, T. B. ,and Birbeck, M. S. C. (1961) J Invest Dermatol 36, 243-252

13. Okazaki et al. (1976) J Invest Dermatol 67, 541-547

14. Seiberg, M (2001) Pigment Cell Res 14, 236-242

15. Cooksey et al. (1997) J Biol Chem 272, 26226-26235

16. Lerner et al (1949.) J Biol Chem 178, 185-195

17. Rodriguez-Lopez, et al (2001) Biochim Biophys Acta 1548, 238-256.

18. Tripathi, et al (1992) J Biol Chem 267, 23707-23712

19. del Marmol et al. (1996) FEBS Lett 381, 165-168

20. Oetting, W. S. (2000) Pigment Cell Res 13, 320-325

21. Korner, A, and Pawelek, J (1982) Science 217,1163-1165

22. Kondoh, et al. (1996) Zoolog Sci 13, 825-831

23. Olivares et al (2001) Biochem J 354, 131-139

24. Kwon et al. (1987) Proc Natl Acad Sci USA 84, 7473-7477

25. Kwon et al. (1988) Biochem Biophys Res Conunun 153, 1301-1309.

26. Muller et al. (1988) Embo J 7, 2723-2730

27. Ruppert, et al. (1988) Embo J 7, 2715-2722

28. Yamamoto, et al. (1987) Jpn J Genet 62, 271-274

29. Jackson and Bennett (1990) Proc Natl Acad Sci USA 87, 7010-7014

30. Spritz, R. A. (1994) Hum Mol Genet 3, 1469-1475

31. Yokoyama, et al. (1990) Nucleic Acids Res 18, 7293-7298

32. Bloch, B , and Ryhiner, P (1917) Exp Med 5, 179

33. Lerner, A. B. , and Hendee, J. R., Jr (1973) J Invest Dermatol 60, 16-19

34. Stevens, A, and Chalk, B. T. (1996) Pigments and minerals In “Theory and Practice of Histological Techniques” (J D Bancroft, and A Ste ens, eds), pp 243-267, Churchill Li ingstone, N.Y.

35. Bobrow et al. (1989) J Imnmunol Methods 125, 279-285

36. Weiner, L, and Green, H (1998) Differentiation 63, 263-272

37. Cabanes, J, Chazarra, S , and Garcia-Carmona, F (1994) J Pharm Pharmacol 46, 982-985.

38. Le et al. (1994) Crit Rev Oncog 5, 141-168

39. Lindquist et al. (1998) Exp Eye Res 67, 259-264

40. Fietz et al. (1993) J Cell Biol 121, 855-865

41. Rothnagel, J. A., and Rogers, G. E. (1986) J Cell Biol 102, 1419-1429

42. Nakajima et at. (1982) Am J Surg Pathol 6, 715-727.

43. Kahn and Pomerantz (1980) Phytochemistry 19, 379-385

44. Ros et al (1994) Biochim Biophys Acta 1204, 33-42

45. Wichers et al. (1984) Planta 162, 334-341

46. Beermann et al. (1992) Proc Natl Acad Sci USA 89, 2809-2813

47. Barsh, G. S. (1996) Trends Genet 12, 299-305

48. Sturm et al. (2001) Gene 277, 49-62

49. Holstein et al. (1973) Yale J Biol Med 46, 560-571

50. Bouchard et al. (1989) J Exp Med 169, 2029-2042.

51. Winder (1991) Biochem Biophys Res Commun 178, 739-745

52. Winder et al. (1993) J Cell Sci 104, 467-475

A number of embodiments of the invention have been described. Nevertheless, it will be understood that various modifications may be made without departing from the spirit and scope of the invention. Accordingly, other embodiments are within the scope of the following claims.

1. A method of detecting tyrosinase in a cell or tissue sample, comprising:
contacting a cell or tissue sample with a tyrosinase substrate, said substrate being coupled, directly or indirectly, to a label; and
detecting the presence of the labeled substrate bound to tyrosinase or another molecule of the sample,
thereby detecting tyrosinase in a cell or tissue sample.

2. The method of claim 1, wherein the sample is a skin cell or tissue.

3. The method of claim 1, wherein the sample is an eye cell or tissue.

4. The method of claim 1, wherein the sample is a blood tissue.

5. The method of claim 1, wherein the sample is a lymph tissue.

6. The method of claim 1, wherein the tyrosinase substrate is tyrosine or a tyrosine analog.

7. The method of claim 1, wherein the tyrosinase substrate is tyramide.

8. The method of claim 1, wherein the tyrosinase substrate is DOPA.

9. The method of claim 1, wherein the tyrosinase substrate is coupled to first member of a specific binding pair and the label is coupled to a second member of a specific binding pair.

10. The method of claim 9, wherein the first and second members of the specific binding pair are biotin and streptavidin.

11. The method of claim 9, wherein the first and second members of the specific binding pair are antigen and antigen-specific antibody.

12. The method of claim 1, wherein the label is a fluorescent label.

13. The method of claim 1, wherein the sample is a frozen section.

14. The method of claim 1, wherein the sample is unfixed or fixed in one or more of: methanol, acetone, and formaldehyde.

15. A method of detecting tyrosinase in a sample, comprising:
contacting the sample with a biotinylated tyrosinase substrate;
contacting the sample with streptavidin conjugated to a label; and
detecting the presence of the label,
thereby detecting tyrosinase in a sample.

16. The method of claim 15, wherein the sample is a skin cell or tissue.

17. The method of claim 15, wherein the sample is an eye cell or tissue.

18. The method of claim 15, wherein the sample is a blood tissue.

19. The method of claim 15, wherein the sample is a lymph tissue.

20. The method of claim 15, wherein the tyrosinase substrate is tyrosine or a tyrosine analog.

21. The method of claim 15, wherein the tyrosinase substrate is tyramide.

22. The method of claim 15, wherein the tyrosinase substrate is DOPA.

23. The method of claim 15, wherein the label is a fluorescent label.

24. The method of claim 15, wherein the sample is a frozen section.

25. The method of claim 1, wherein the sample is unfixed or fixed in one or more of: methanol, acetone, and formaldehyde.

26. A method of identifying a compound that modulates pigmentation, the method comprising:
contacting a cell or tissue with a test compound;
contacting the cell or tissue with a tyrosinase substrate, wherein the tyrosinase substrate is coupled directly or indirectly to a label;
detecting the label in the cell or tissue; and
selecting the test compound as a compound that modulates pigmentation if the amount, localization, or distribution of the label in the presence of the test compound differs from the amount, localization, or distribution of the label in the absence of the test compound,
thereby identifying a compound that modulates pigmentation.

27. The method of claim 26, further comprising the step of contacting the cell or tissue with UV radiation.

28. The method of claim 26, wherein the cell or tissue is a skin cell or tissue.

29. The method of claim 26, wherein the cell or tissue is hair.

30. The method of claim 26, wherein the cell or tissue is an eye cell or tissue.

31. The method of claim 26, wherein the tyrosinase substrate is tyrosine or a tyrosine analog.

32. The method of claim 26, wherein the tyrosinase substrate is tyramide.

33. The method of claim 26, wherein the tyrosinase substrate is DOPA.

34. The method of claim 26, wherein the tyrosinase substrate is coupled to biotin and the label is coupled to streptavidin.

35. The method of claim 26, wherein the label is a fluorescent label.

36. The method of claim 26, further compromising the step of testing the selected compound in vivo on an animal.

37. The method of claim 26, wherein the selected compound is a skin, hair or eye bleaching agent.

38. The method of claim 26, wherein the selected compound is a skin, hair or eye darkening agent.

39. The method of claim 27, wherein the selected compound is a sunscreen.

40. A method of identifying a compound that modulates pigmentation, the method comprising:
contacting a cultured melanocyte with a test compound;
contacting the cultured melanocyte with a tyrosinase substrate, wherein the tyrosinase substrate is coupled directly or indirectly to a label;
detecting the label in the cultured melanocyte; and
selecting the test compound as a compound that modulates pigmentation if the amount, localization, or distribution of the label in the presence of the test compound differs from the amount, localization, or distribution of the label in the absence of the test compound,
thereby identifying a compound that modulates pigmentation.

41. The method of claim 40, wherein the test compound is a small molecule.
